(19) United States
(12) Patent Application Publication
(10) Pub. No.: US 2007/0208241 A1
(43) Pub. Date: Sep. 6, 2007

(54) Vital Sign Sensing Device

(76) Inventor: Marc Drucker, Newton, MA (US)

Correspondence Address: HESLIN ROTHENBERG FARLEY & MESITI PC, 5 COLUMBIA CIRCLE, ALBANY, NY, 12203, US

(21) Appl. No.: 11/668,772

(22) Filed: Jan. 30, 2007

Related U.S. Application Data
(60) Provisional application No. 60/763,641, filed on Jan. 31, 2006.

(51) Int. Cl. A61B 5/00; A61B 5/02; A61B 5/04; A61B 5/05

(52) U.S. Cl. 600/323; 600/549; 600/547; 600/365; 600/316; 600/509; 600/500

(57) ABSTRACT

Vital sign sensing device includes a housing adapted to support a hand of a user and sensors adapted to detect vital signs of the user. The sensor device includes an arithmetic processor adapted to receive the electrical signal, manipulate the signal, and generate an electrical signal representative of the vital signs. The vital sign sensing device can be incorporated into various structures and equipment, such as, an armrest or seating device, a stretcher, or a kiosk.

### CROSS-REFERENCES TO RELATED APPLICATIONS

This application claims priority from co-pending U.S. provisional patent application No. 60/763,641, filed Jan. 31, 2006, which is hereby incorporated in its entirety by reference.

### TECHNICAL FIELD

This invention relates to a vital sign sensing device adapted to measure a multitude of vital signs. The vital sign sensing device can collect, analyze and relay vital sign data.

### BACKGROUND OF THE INVENTION

The efficient detection and monitoring of a patient's vital signs is critical to ensuring proper treatment. In most health care settings, patients are faced with long wait times and overcrowded waiting rooms. Assessing a patient's vital signs is expensive and time consuming. Healthcare workers benefit from tools and equipment that improve their efficiency. The present invention has the ability to revolutionize the monitoring and treatment of patients by allowing for the collection and analysis of vital sign measurements, without the necessary assistance of a healthcare provider.

The vital sign sensing device of the present invention can aid the medical profession to more efficiently and effectively assist patients by reducing care givers' workloads resulting in reducing the time patients spend waiting for care. In the US, the average patient spends about an hour and a half waiting to be seen by a medical professional. Likewise, the process of admitting patients and measuring vital signs requires, on average, an additional 35 minutes. As a result of long waiting periods, patient are less satisfied, and those that require immediate attention often suffer.

### SUMMARY OF THE INVENTION

According to aspects of the present invention, the vital sign sensing device is patient-friendly and easy to use. Some aspects include the ability to measure a multitude of physiological indicators, collect and analyze the physiological data and relay the data to remote locations. The vital sign sensing device embodies multiple sensors adapted to measure vital signs. It also embodies an arithmetic processor adapted to receive vital sign data, manipulate the data, and generate an output based on the data analysis. The present invention also has the capability of measuring, analyzing and communicating a patient's physiological indicators on an ongoing basis and alerting health care providers of medical emergencies. Additional embodiments include an arm cuff for measuring blood pressure and a display screen for displaying data.

The present invention will reduce healthcare costs by enabling more efficient vital sign measurements. The present invention will significant impact hospital procedures, reduce care-givers' workloads and improve a patient's waiting room experience. The multiple and diverse embodiments of the invention provide for applications beyond the emergency room or waiting room setting. The vital sign sensing device is easily mountable and can be mounted or incorporated into any apparatus, such as a chair, kiosk, stretcher, bed, home product, ambulance and more to allow for the safe, easy and efficient monitoring of vital signs. Further, the invention will allow for vital sign measurements to be more easily assessed in less common locations, for example, at-home, on the battlefield and during patient transport.

The present invention will radically change the waiting room experience by allowing the patient to measure, analyzing, and communicate their vital signs without the assistance of a healthcare worker. As part of its functionality, the present invention has the capability to measure a multitude of physiological indicators, analyze the measurements taken and relay the information. Further, the vital sign sensing device can track a patient's vital signs over time, and therefore, can help identify trends. Also, the vital sign data collected can be stored in digital form and can therefore be integrated into existing hospital digital records. The present invention may reduce healthcare costs by reducing caregivers' workloads, enable more efficient patient admitting processes, enable more accurate vital sign measurements, automatically integrate digital patient records, and allow for vital sign measurements to be obtained in diverse settings (for instance, at the hospital, at home, on the battlefield, or at the office).

One embodiment of the invention is a vital sign sensing device that includes a housing adapted to support the hand of the user. The housing includes a cavity, and the cavity is adapted to receive one or more fingers of the user. The cavity may further include a sensor adapted to detect one or more vital signs of the user and to output an electrical signal representative of the vital sign(s). Another aspect of the invention includes a second sensor which may also be mounted in the housing of the vital sign sensing device. The second sensor may be adapted to further contact the arm the user, detect at least one further vital sign of the user, and output an additional electrical signal representative of the further vital sign(s). One embodiment may also include an arithmetic processor adapted to receive electrical signals, manipulate electrical signals, and generate an additional electrical signal representative of one or more of the vital signs.

Another embodiment of the present invention is a vital sign sensing device, which includes a housing, a cavity, and a handgrip. The handgrip may be mounted across a cavity or in any other configuration. The vital sign sensing device includes a sensor mounted in the handgrip. The sensor is adapted to detect one or more vital signs from the user and to output an electrical signal representative of the vital sign(s). An additional feature of the vital sign sensing device includes an arithmetic processor adapted to receive an electrical signal, manipulate the electrical signal, and generate a further electrical signal representative of the vital sign.

An additional embodiment of the invention is the vital sign sensing device as discussed above, incorporated into an arrangement, such as a chair. The arrangement may include a console which may further include an armrest. The vital sign sensing device is positioned in the console and includes one or more sensors adapted to contact the user seated in the seating device. The sensors are adapted to detect one or more vital signs and are adapted to output an electrical signal representative of one or more of the vital signs. Further, the device may include an arithmetic processor as discussed above.

### BRIEF DESCRIPTION OF THE DRAWING

The present invention will be better understood from the detailed description given herein below and the accompanying drawings which are given by way of illustration only, and thus are not a limitative of the present invention and wherein:

FIG. 1 is a perspective view of a vital sign sensing device according to one aspect of the invention.

FIG. 2A is a perspective view of a vital sign sensing device according to another embodiment of the invention.

FIG. 2B is a perspective view of the vital sign sensing device shown in FIG. 2A as used by a user.

FIG. 3 is a perspective view of a vital sign sensing device according to another aspect of the invention.

FIG. 4A is a perspective view of a vital sign sensing device mounted to a freestanding structure according to another aspect of the invention.

FIG. 4B is a perspective view of a vital sign sensing device mounted to a surface according to another aspect of the invention.

FIG. 5A is a perspective view of a vital sign sensing device according to a further embodiment of the invention.

FIG. 5B is a cross-sectional view of the device as shown in FIG. 5A as viewed along section lines 5B-5B in FIG. 5A.

FIG. 6 is a perspective view of a vital sign sensing device according to another aspect of the present invention.

FIG. 7 is a perspective view of a vital sign sensing device according to a further embodiment as associated with a chair.

FIG. 8 is a perspective view of a further embodiment of the present invention shown mounted to a chair.

FIG. 9 is a perspective view of a further embodiment of the present invention.

FIG. 10. is a perspective view of another embodiment of the present invention.

FIG. 11 is a schematic diagram of an electrical system according to a further embodiment of the present invention.

### DETAILED DESCRIPTION OF THE INVENTION

Reference is now made to the above drawings wherein the identical numbers refer to the like parts throughout. It is noted that while the invention is described herein, all such adaptations and alternate embodiments are considered to fall within the scope of the claims appended hereto.

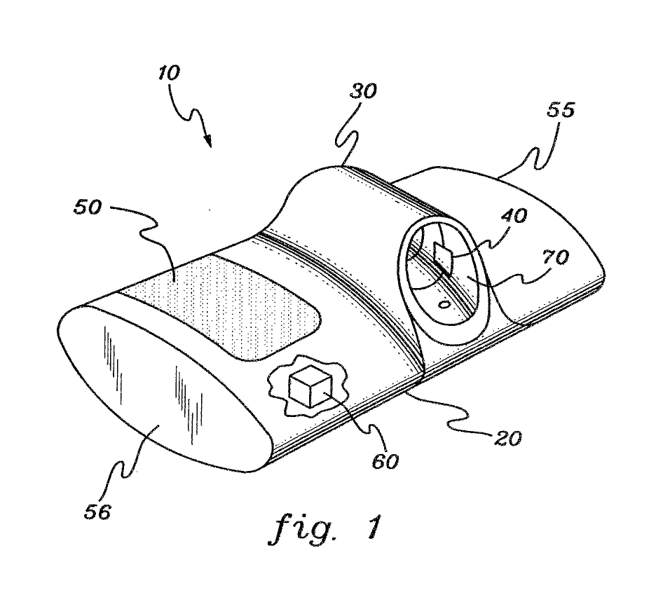

FIG. 1 is a perspective view of a vital sign sensing device 10 according to one aspect of the invention. FIG. 1 shows a vital sign sensing device 10, which includes a housing 20 adapted to support a hand of the user (not shown), and a cavity 30 adapted to receive at least one finger of the user. First sensor 40 may be mounted in any part of cavity 30. Cavity 30 can be cylindrical or non-cylindrical and typically has at least one aperture 70. Aperture 70 may be located anywhere in cavity 30, including, but not limited to, the top and sides of cavity 30. Further, cavity 30 may comprise a first cylindrical section and a second cylindrical section, where the first cylindrical section may be pivotally mounted to the second cylindrical section. For example, the first section is a cylindrical tube pivotally mounted to the second section which is also a cylindrical tube. Cavity 30 also may be adjustable to fit the finger of the user and may receive any finger of the hand. Further, multiple sensors may be mounted in cavity 30.

According to one aspect of the invention, at least one first sensor 40 may be mounted in cavity 30. First sensor 40 may be adapted to detect one or more vital signs of the user and to output at least one electrical signal, for example, a first electrical signal, representative of the vital sign(s). First sensor 40 may be adapted to include a physiological indicator sensor, which may include one or more of a pulse rate sensor, a pulse oxygenation sensor, a temperature sensor, an EKG sensor, a blood glucose sensor, a basal metabolism sensor, a body mass index sensor, a body fat sensor, or any other type of physiological sensor.

As shown in FIG. 1, Housing 20 may include a second sensor 50 adapted to contact the hand of the user. Second sensor 50 may be adapted to detect one or more vital signs of the user and to output an electrical signal, for example, a second electrical signal, representative of the vital sign(s). Second sensor 50 may be adapted to include a physiological indicator sensor, which may include one or more of a pulse rate sensor, a pulse oxygenation sensor, a temperature sensor, an EKG sensor, a blood glucose sensor, a basal metabolism sensor, a body mass index sensor, a body fat sensor, or any other type of physiological sensor. Second sensor 50 may be located anywhere on housing 20 or in cavity 30. Either first sensor 40 or second sensor 50 or both first sensor 40 and second sensor 50 may be contacted by the hand of the user for device 10 to measure vital sign(s). Additionally, housing 20 may include a first end 55 and a second end 56. Second sensor 50 may be located at or adjacent to first end 55 or second end 56, or in between first end 55 and second end 56. Further, cavity 30 may be located at or adjacent to either first end 55 or second end 56, or, as shown in FIG. 1, cavity 30 may be located in between first end 55 and second end 56.

According to one aspect of the invention, the vital sign sensing device 10 may further include one or more additional sensors adapted to detect additional vital signs of the user and to output further electrical signals, for example, a third or a fourth electrical signal output, representative of at least one vital sign. The additional sensor(s) may contact any portion of the hand of the patient (not shown) and may be adapted to include a physiological indicator sensor which may include one or more of a pulse rate sensor, a pulse oxygenation sensor, a temperature sensor, an EKG sensor, a blood glucose sensor, a basal metabolism sensor, a body mass index sensor, a body fat sensor, or any other type of physiological sensor. The additional sensor(s) may be incorporated in any one or more embodiments of the present invention disclosed herein.

As shown in FIG. 1, the vital sign sensing device 10 may further include one or more arithmetic processors 60 positioned in housing 20. Arithmetic processor 60 may be adapted to receive electrical signals, such as a first, second, or fourth electrical signal, manipulate the electrical signals, and generate an additional electrical signal, for example a third electrical signal output, representative of one or more vital signs. The electrical signals can be one of a wired or wireless signal. The wireless signal may include an RFID signal, a WIFI signal, a cellular signal, or other related signal. Arithmetic processor 60 may be adapted to store one or more electrical signals corresponding to one or more vital signs and may further be adapted to record the electrical signals for one or more time periods. The terminology describing first, second, third and fourth electrical signals and their relationships is common in all aspects of the invention disclosed herein.

An additional aspect of vital sign sensing device 10 may include a means for communicating with an information system, for instance, a healthcare information system. Also, vital sign sensing device 10 may further comprise a finger print reader or other identification indicator. A finger print reader or other identification indicator may be incorporated into any embodiment of the invention disclosed herein.

Additionally, vital sign sensing device 10 may also include a source of power adapted to supply the physiological sensors and the arithmetic processor 60 with power. The source of power may include electric power, solar power, battery power, or other source of power.

Vital sign sensing device 10 is mountable to various structures. For instance, vital sign sensing device 10 can be mounted to handles, poles, bars, tables, and counters, among other structures. Vital sign sensing device 10 may also be easily mounted and dismounted to any of these structures, for example, by conventional fasteners (not shown). The device 10 may also be mounted so device 10 can be rotated. Vital sign sensing device 10 may also be incorporated into sports equipment, medical equipment, office equipment, security products, and furniture, among other equipment.

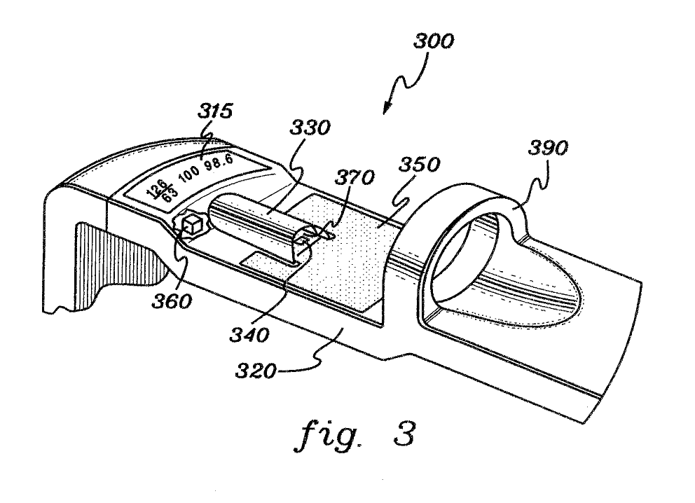

Further, vital sign sensing device 10 may include an arm cuff (not shown), for example, as shown and described in FIG. 3 below. The arm cuff may be adapted to detect one or vital signs, including blood pressure and pulse rate or any other physiological indicator that can be detected on the arm of the user. The arm of the user is intended to broadly encompass the fingers, hand, wrist, forearm, bicep and upper region of the arm. The arm-cuff may be adjustable to fit the arm of a user and may be made out of any type of material, rigid or flexible.

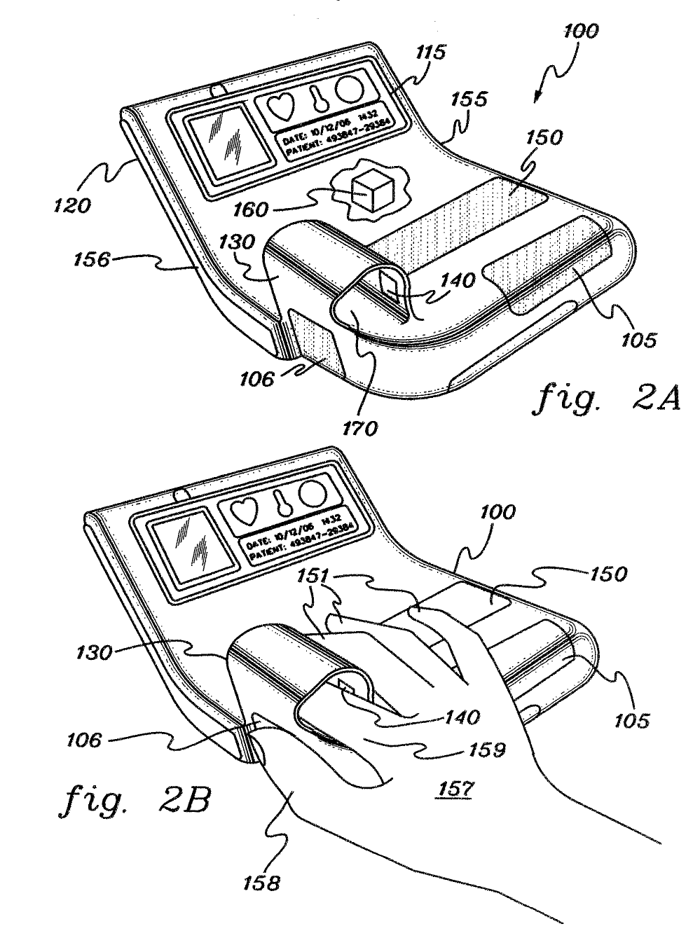

FIG. 2A is a perspective view of vital sign sensing device 100 according to another embodiment of the invention. As shown in FIG. 2A, vital sign sensing device 100 includes a housing 120 for supporting the hand of a user (not shown), and a cavity 130 for supporting one or more fingers of the user (not shown). According to one aspect of the invention, cavity 130 may be cylindrical or non-cylindrical and may include aperture 170. Aperture 170 may be located anywhere on cavity 130, including, but not limited to, the top and sides of cavity 130. Cavity 130 may include a first section and a second section and the first section may be pivotally mounted to the second section, as described above. Cavity 130 may be adjustable to fit the finger(s) of the user (not shown). Further, cavity 130 may receive any finger(s) of the hand of the user and multiple sensors may be mounted in cavity 130.

According to one aspect of the invention as shown in FIG. 2A, a first sensor 140 may be located anywhere in cavity 130. First sensor 140 may be adapted to detect at least one vital sign of the user (not shown) and to output at least one electrical signal representative of the vital sign(s). First sensor 140 includes a physiological indicator sensor, which may include one or more of a pulse rate sensor, a pulse oxygenation sensor, a temperature sensor, an EKG sensor, a blood glucose sensor, a basal metabolism sensor, a body mass index sensor, a body fat sensor, or any other type of physiological sensor.

Further, housing 120 may also include a second sensor 150, and second sensor 150 may be adapted to contact the hand of the user (not shown). Second sensor 150 may be adapted to detect one or more vital signs of the user and to output an electrical signal representative of the vital sign(s). Second sensor 150 may include a physiological indicator sensor, which may include one or more of a pulse rate sensor, a pulse oxygenation sensor, a temperature sensor, an EKG sensor, a blood glucose sensor, a basal metabolism sensor, a body mass index sensor, a body fat sensor, or any other type of physiological sensor. Second sensor 150 may be located anywhere in housing 120 or cavity 130. Either first sensor 140 or second sensor 150 may be contacted for device 100 to function. Housing 120 may include first end 155 and second end 156 and the second sensor 150, may be positioned at or adjacent to first end 155 and second end 156 or in between first end 155 and second end 156. As shown in FIG. 2A, second sensor 150 may be positioned at or adjacent to first end 155. Further, cavity 130 may be located at or adjacent to either first end 155 or second end 156 or in between first end 155 and second end 156. As shown in FIG. 2A, cavity 130 is located at first end 155.

As shown in FIG. 2A, device 100 may comprise at least one third sensor 105 adapted to detect one or more vital signs of the user and to output one or more electrical signals representative of the vital signs. Vital sign sensing device 100 may include more than three sensors. Third sensor 105 may contact any portion of the hand of the patient. Third sensor 105 may include a physiological indicator sensor, as described above. At least one third sensor 105 may be incorporated in any embodiments of the invention disclosed herein.

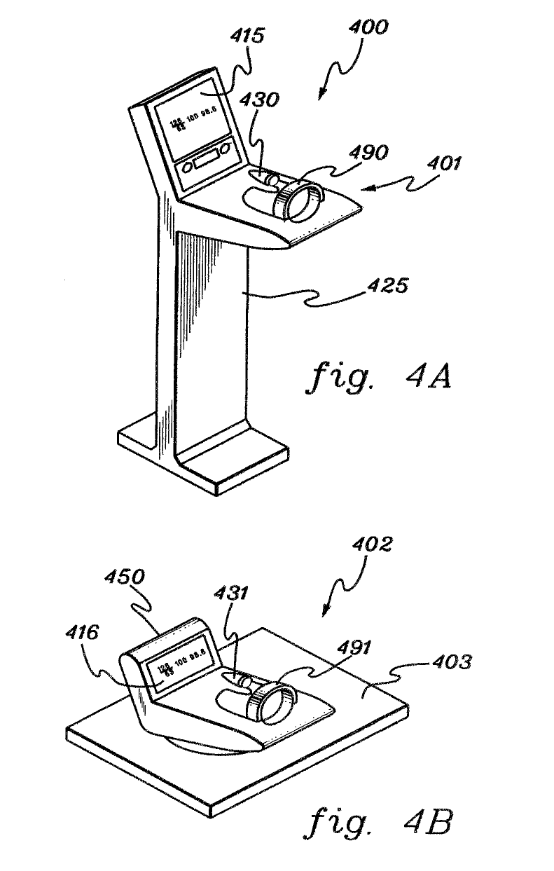

An additional aspect of the invention may include a fourth sensor 106 adapted to detect a vital sign of the user (not shown) and to output an electrical signal representative of a vital sign. Vital sign sensing device 100 may include more than four sensors. Fourth sensor 106 may contact any portion of the hand of the patient, for example, the thumb of a patient as shown in FIG. 4B. Fourth sensor 106 may include a physiological indicator sensor discussed above. Fourth sensor 106 may be incorporated in any embodiments of the present invention disclosed herein.

As shown in FIG. 2A, vital sign sensing device 100 may further comprise an arithmetic processor 160 adapted to receive one or more electrical signals, manipulate electrical signals, and generate an additional electrical signal(s) representative of a vital sign(s). The electrical signals can be a wired or wireless as discussed above. Arithmetic processor 160 may be adapted to store one or more electrical signals corresponding to a vital sign(s) and is adapted to record the electrical signal(s) for one or more time periods. Vital sign sensing device 100 may further includes a means for communicating with an information system(s), including, but not limited to, a healthcare information system. Vital sign sensing device 100 may further include a finger print reader or other identification indicator.

Additionally, vital sign sensing device 100 may include a source of power adapted to supply sensors 140, 150, 105, and 106 and arithmetic processor 160 with power. The source of power may include electric power, solar power, battery power, or other source of power.

In contrast to device 10 as shown in FIG. 1, device 100 is adapted to include a display screen 115 mounted to housing 120. Display screen 115 is adapted to receive input from arithmetic processor 160, a human operator, and an external interface. Display screen 115 can include one or more of a touchpad and a keypad and any conventional means for inputting information. Display screen 115 may display data and an electrical signal representative of one or more vital signs, and display screen 115 may display information received from a remote location, such as a nursing station. Further, display screen 115 may include multiple display screens and may be mounted in housing 120 to separate from housing 120, and may display information provided by a hard-wired communication, a radio-frequency communication, optical communication, or any other type of communication. Information displayed on display screen 115 may be communicated to a printer either mounted in the housing 120 or remote to the housing 120. Display screen 115 may be adapted to tilt or move in any manner to allow for optimal viewing of display screen by user or healthcare provider (not shown). This aspect may be incorporated into any embodiments of the present invention disclosed herein. Housing 120 may also be able to adjust to allow for more comfortable viewing of display screen 115. For instance, housing 120 may include hinges, or other movable structures, extending the length of the housing between first section 155 and second section 156 to allow for housing 120 to adjust. This aspect may be incorporated into any embodiments of the present invention disclosed herein.

In another aspect of the invention, vital sign sensing device 100 may be mounted to a broad variety of structures. For instance, vital sign sensing device 100 may be mounted to handles, poles, bars, tables, and counters, among other structures. Vital sign sensing device 100 may be incorporated into sports equipment, medical equipment, office equipment, security products, and furniture among other structures. Vital sign sensing device 100 can be easily mounted and dismounted to any of these structures, for example by way of conventional fasteners. Device 100 may rotate when mounted.

Further, vital sign sensing device 100 may include an arm cuff (not shown). The arm-cuff is adapted to detect at least one of blood pressure and pulse rate or any other physiological indicator that can be detected on the arm of the user. The arm of the user (not shown) is intended to broadly include the fingers, hand, wrist, forearm, bicep and upper region of the arm. The arm-cuff may be adjustable to fit the arm of a user.

FIG. 2B is a further perspective view of a vital sign sensing device 100 shown in FIG. 2A as used by a user as represented by hand of user 157. Pointer finger of user 159 is inserted into cavity 130. Any finger may contact any of sensors 140, 150, 105 and 106. Second sensor 150 may be contacted by one or more fingers of user 151. Third sensor 105 may be contacted by any part of the hand of the user 157.

FIG. 3 is a perspective view of a vital sign sensing device 300 according to another aspect of the invention. As shown in FIG. 3, the vital sign sensing device 300 includes a housing 320 adapted to support the hand of the user (not shown), and a cavity 330 adapted to receive one or more fingers of the user (not shown).

At least a first sensor 340 and a second sensor 350 may be mounted anywhere in cavity 330. First sensor 340 and second sensor 350 may comprise any of the physiological indicator sensors as disclosed above. Cavity 330 can be cylindrical or non-cylindrical and may have at least one open first aperture 370. As discussed with respect to other aspects of the invention, aperture 370 may be located anywhere on cavity 330, including but not limited to the top and sides of the cavity 330. Cavity 330 may comprise a first section and a second section, wherein the first section is pivotally mounted to the second section as discussed above. Cavity 330 may be adjustable to fit the finger(s) of the user. Cavity 330 may receive any finger(s) of the user. Multiple physiological sensors may be mounted in cavity 330. The third and fourth sensors may also be provided in device 300.

As shown in FIG. 3, vital sign sensing device 300 may further include an arm cuff 390 mounted to housing 320. Arm cuff 390 may be adapted to detect at least one of blood pressure and pulse rate or any other physiological indicator that can be detected on the arm of the user (not shown). The arm of the user is broadly intended to include the fingers, hand, wrist, forearm, bicep and upper region of the arm. Arm cuff 390 may be adjustable to fit the arm of a user.

As shown in FIG. 3, as is typical of other aspects of the invention, vital sign sensing device 300 further comprises arithmetic processor 360 adapted to receive, manipulate, and generate electrical signals representative of vital signs as discussed above.

Vital sign sensing device 300 may further comprise a source of power as discussed above. Vital sign sensing device 300 may also comprise a display screen 315 mounted to housing 320. Similar to display screen 215 in FIG. 2A, display screen 315 is adapted to receive input and display data, for example physiological data of the user. Display screen 315 may be adapted to move and adjust for more comfortable viewing of data displayed on display screen 315, as discussed above.

Further, vital sign sensing device 300 may be mounted. For instance, vital sign sensing device 300 can be mounted to handles, poles, bars, tables, counters, etc. Vital sign sensing device 300 can be incorporated into sports equipment, medical equipment, office equipment, security products, and furniture, among other equipment and related products. Vital sign sensing device 300 can be easily mounted dismounted and rotated as discussed above.

FIG. 4A is a perspective view of an apparatus 400 incorporating a vital sign sensing device 401 mounted to a freestanding structure, for example, a kiosk. Vital sign sensing device 401 may be mounted, for instance, to free a standing structure 425, a handle, a pole, a bar, a table, a kiosk, and a counter, among other structures. Vital sign sensing device 401 may be incorporated into sports equipment, medical equipment, office equipment, security products, and furniture, among other equipment or products. Likewise, vital sign sensing device 401 can be easily mounted and dismounted to any of these structures, for example, by conventional fastners. Also, vital sign sensing device 401 may be mounted in a manner to allow device 401 to rotate. The vital sign sensing device 401 can incorporate any embodiments of the invention. Vital sign sensing device 401 may be similar to vital sign sensing device 100 and vital sign sensing device 300 as shown in FIG. 2A and FIG. 3 respectively and may incorporate any of the features of vital sign sensing device 100 and vital sign sensing device 300. Vital sign sensing device 401 may include cavity 430, and display screen 415, similar to cavity 130 and display screen 115 as shown in FIG. 2A. Vital sign sensing device 401 may also include arm cuff 490, similar to arm cuff 390 as disclosed in FIG. 3.

FIG. 4B is a perspective view of apparatus 402 incorporating a vital sign sensing device 450 mounted to a surface 403, for example, a table top. Similar to device 401 shown in FIG. 4A, vital sign sensing device 450 may be similar to vital sign sensing device 100 and vital sign sensing device 300 as shown in FIG. 2A and FIG. 3 and may incorporate all of the features of vital sign sensing device 100 and vital sign sensing device 300, including but not limited arm cuff 491, cavity 431 and display screen 416.

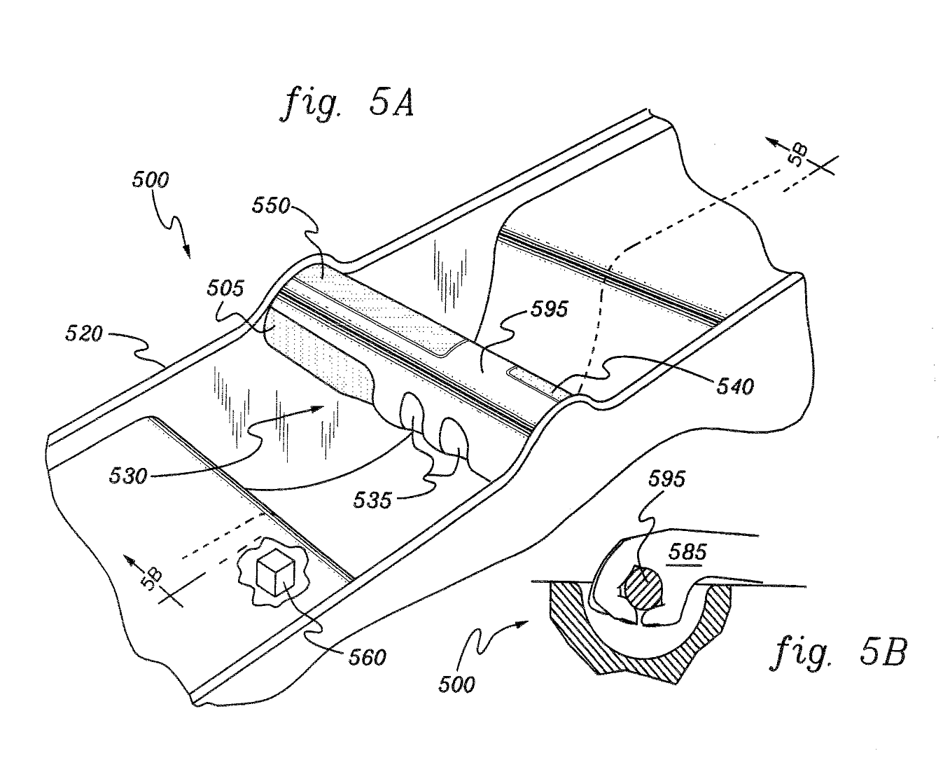

FIG. 5A is a perspective view of vital sign sensing device 500 according to a further embodiment of the invention. As shown in FIG. 5A, vital sign sensing device 500 includes a housing 520 for supporting the hand of the user (not shown), a hand grip 595 and a cavity 530 to provide space for user to comfortable and easily grip handgrip 595.

According to FIG. 5A, a first sensor 540 may be mounted in handgrip 595. Handgrip 595 may be cylindrical or non-cylindrical and may be mounted across cavity 530. Handgrip 595 may be mounted on either or both sides of cavity 530, or may be supported in the center of cavity 530 by a pedestal or post (not shown). Handgrip 595 may be mounted across the cavity 530 as shown in FIG. 5A, or may extend vertically from cavity 530, for example in a fashion similar to a flight controller stick. First sensor 540 is adapted to detect one ore more vital signs of the user and to output one or more electrical signals representative of the vital sign(s), as discussed above. Additional sensors may be mounted in cavity 530 and in handgrip 595, for example, a sensor 505 and a sensor 550 may be provided on handgrip 598 or anywhere on or in housing 520 and may include aspects of first sensor, as discussed above.

As shown in FIG. 5A, handgrip 595 may further include finger indentations 535 to guide the user's fingers (not shown) into placement on handgrip 595 and for ease of gripping. Further, according to FIG. 5A, vital sign sensing device 500 may include at least one arithmetic processor 560 adapted to receive, manipulate and generate electrical signals representative of one or more vital signs, as discussed above.

Vital sign sensing device 500 further comprises a means for communicating with an information system (not shown). Vital sign sensing device 500 may further include a finger print reader (not shown) or other identification indicator (not shown) and vital sign sensing device 500 may further include a source of power adapted to supply the physiological sensors and the arithmetic processor 560 with power. The source of power may include electric power, solar power, battery power, or other source of power.

Vital sign sensing device 500 may be mounted, as discussed above. Additionally, vital sign sensing device 500 can include two or more handgrips, each with or without a separate housing 520. Device 500 may further include a finger print reader or other identification indicator.

FIG. 5B is a cross-sectional view of the vital sign sensing device 500 as shown in FIG. 5A as viewed along section lines 5B-5B. FIG. 5B shows handgrip 595 of another sensing device 500 being gripped by hand of a user 585.

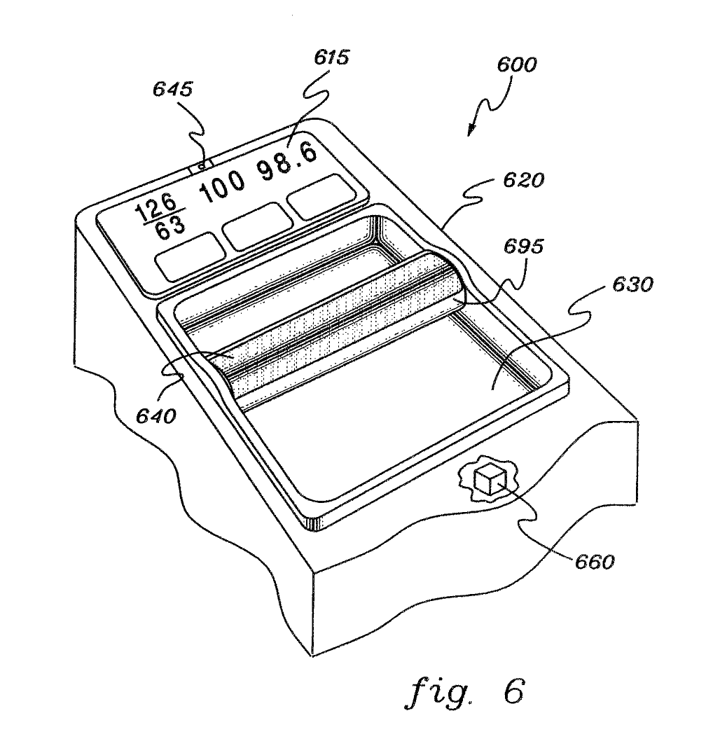

FIG. 6 is a perspective view of another vital sign sensing device 600, similar to device 500 as shown in FIG. 5A and FIG. 5B. As shown in FIG. 6, vital sign sensing device 600 includes a housing 620 for supporting the hand of the user (not shown), a handgrip 695 and a cavity 630 to provide space for hand of the user to comfortably and easily grip handgrip 695. According to FIG. 6, first sensor 640 is mounted in handgrip 695. Handgrip 695 can be cylindrical or non-cylindrical and may be mounted as described above. First sensor 640 is adapted to detect one or more vital signs of the user and to output at least one electrical signal representative of the vital sign(s). First sensor 640 may include a physiological indicator sensor as described above. Additional sensors may be mounted in handgrip 630.

Vital sign sensing device 600, as shown in FIG. 6, may further include one or more of audio visual mechanism 645, including, but no limited to, a video camera adapted to transmit video images, a speaker adapted to transmit audio speech, and/or a microphone adapted to receive audio speech. Audio visual mechanism 645 may also consist of a palpable mechanism. A palpable mechanism may include a mechanism other than an audio or visual mechanism, for example, a mechanism that vibrates. Vital sign sensing device 600 may include a means for transmitting one or more of a video image or audio speech to a remote location. Audio visual mechanism 645 may be part of any embodiment of the invention described herein.

Further, vital sign sensing device 600 may be mounted. For instance, vital sign sensing device 600 can be mounted to handles, poles, bars, tables, counters, and the like. Vital sign sensing device 600 can be incorporated into sports equipment, medical equipment, office equipment, security products, and furniture, among other equipment or products. Vital sign sensing device 600 can be easily mounted and removed and can additionally have the ability to rotate when mounted. Also, vital sign sensing device 600 can include two or more handgrips, each with or without a separate housing 620.

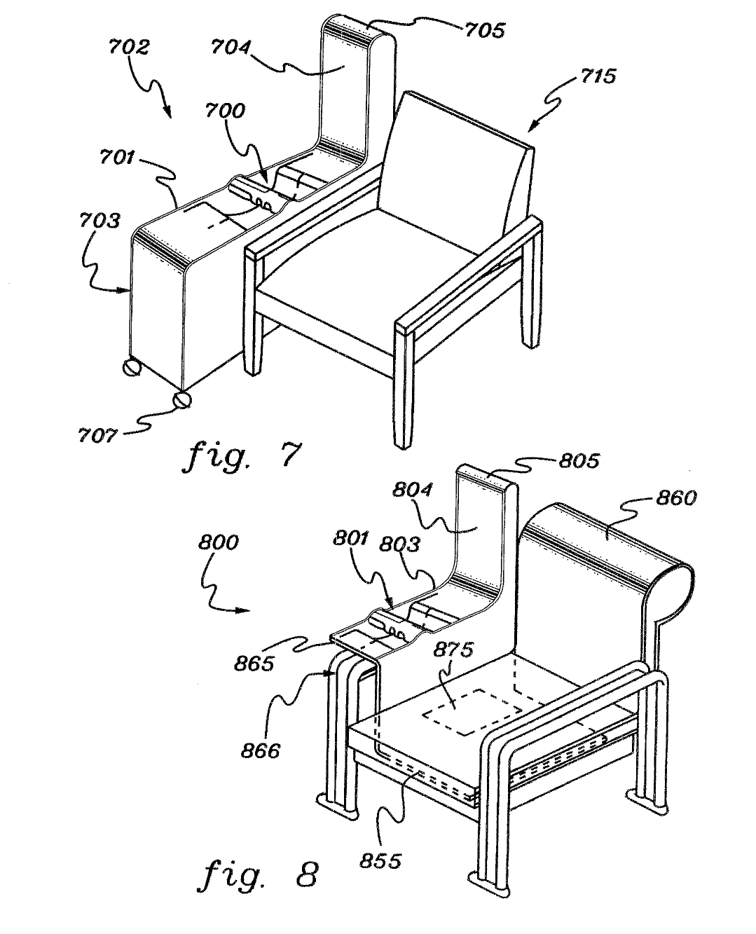

FIG. 7 is a perspective view of an arrangement 702. Arrangement 701 includes a console 703 having a vital sign sensing device 700 adjacent to a chair 715. As shown in FIG. 7, console 703 includes and arm rest 701. Vital sign sensing device 700 includes any of the embodiments of the present invention disclosure herein, and is similar to vital sign sensing device 500 as shown in FIG. 5A. Console 703 may be portable and may have wheels 707 for easy mobility and stationing. Wheels 707 may be adapted to lock and unlock. Console 703 can be placed next to a seating apparatus, such as chair 715, a bench, a couch, or next to a bed, a stretcher, or other apparatus. Console 703 may include a means for adjusting height. For instance, the height of console 703 may be adjusted so that arm rest 701 is at a comfortable and convenient height in relation to chair 715. Console 703 may further include a tower assembly 704 which may include an emergency beacon 705 adapted to receive an electrical signal from the arithmetic processor (not shown). Emergency beacon 705 may be programmed to emit one or more visual, audio or palpable signals if a particular physiological indicator has reached a specified level or measurement.

FIG. 8 is a perspective view of a further arrangement 800 according to a further embodiment of the present invention having a console 803 mounted to a seating device 860 having a vital sign sensing device 801. Console 803 may be adapted to mount to any apparatus, including, but not limited to, seating device 860, a bench, a couch, a bed, a stretcher, or other apparatus. In this aspect, console 803 includes platform 855 adapted to be placed beneath or on top of the seat or applicable place of seating device 860, and an arm rest 865 positioned to support a user's arm (not shown). Arm rest 865 may be placed on arm rest of chair 866 or arm rest 865 may act as an arm rest to seating device 860 if seating device 860 does not have an arm rest 866. Vital sign sensing device 801 may be incorporated in the arm rest 865 of the console 803, but may be positioned anywhere on console 803.

In a further aspect of the invention, console 803 can also include multiple arm rests 865 and multiple vital sign sensing devices 801. Vital sign sensing device 801 may include any embodiments of the present invention disclosed herein. Further, console 803 may be adjustable in anyway possible and may be made of a flexible or rigid material. Platform 855 may comprise at least one platform sensor 875 adapted to detect at least one of pressure, weight, movement, ingress, and egress of the user. Platform sensor 875 may include a physiological indicator as discussed above.

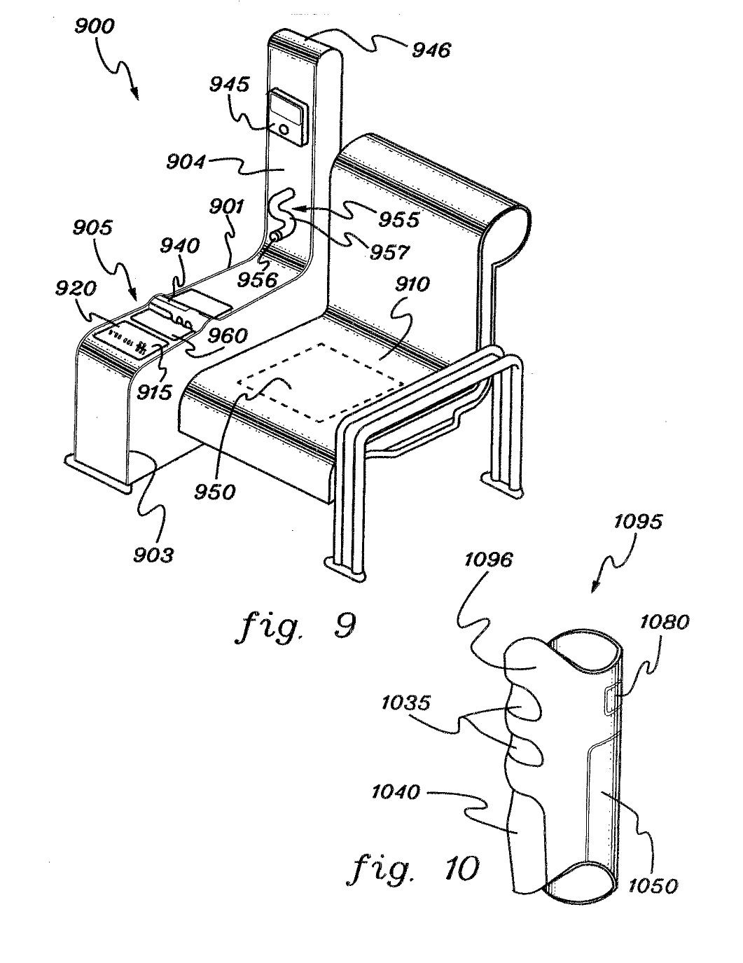

FIG. 9 shows a perspective view of another arrangement 900 of the present invention. According to FIG. 9, arrangement 900 includes a console 903 having a vital sign sensing device 905 and further positioned in arm rest 901. First sensor 940 may be positioned in console 903 and may further positioned in vital sign sensing device 905. Vital sign sensing device 905 may include any embodiments of the present invention, including one or more sensors. First sensor 940 may detect at least one vital sign and may output at least one electrical signal representation of the vital sign. First sensor 940 may include one or more of a pulse rate sensor, a pulse oxygenation sensor, a temperature sensor, an EKG sensor, a blood glucose sensor, a basal metabolism sensor, a body mass index sensor, a body fat sensor, or other physiological related sensor.

As shown in FIG. 9, arrangement 900 further includes seat 910. A second sensor 950 may be mounted in seat 910, but may also be mounted anywhere on seating device 900. Second sensor 950 is adapted to contact the user (not shown) seated in arrangement 900 and second sensor 950 may detect one or more vital signs and may further output at least one electrical signal representative of one or more vital signs. Second sensor 950 may be adapted to detect one or more of movement, respiration, heartbeat, position changes of the user, or other physiological related indicator. Arrangement 900 may contain more than two sensors, which may be located anywhere on the arrangement 900. Arithmetic processor 960 may be mounted in arm rest 901 or anywhere in arrangement 900 and further mounted in the vital sign sensing device 905 and includes all embodiments discussed above.

In a further aspect of the invention, vital sign sensing device 905 further comprises a means for communicating with an information system, including but not limited to a healthcare information system. Identification indicator (not shown).

Arrangement 900 may further include a source of power adapted to supply the physiological sensors and arithmetic processor 960 with power. The source of power may include one or more of electric power, solar power, battery power, or other source of power or a combination thereof, and may be located anywhere on or in arrangement 900.

Further, vital sign sensing device 905 may also include one or more display screens 915 adapted to be mounted anywhere on housing 920 of vital sign sensing device 905. Display screen 915 may be similar to and have all of the functionalities of display screen shown in FIG. 2A and may be mounted any where on console 903.

Seating device 900 is further adapted to include a means 955 for analyzing the breath of a user (not shown), for example a sensor 956 mounted on an arm or rigid cable 957. Means 955 may include a chemical analyzer adapted to identify markers of one or more of oxidative stress and disease or other physiological related indicators. Means 955 for analyzing the breath of a user (not shown) may be incorporated into any embodiment of the invention disclosed herein.

According to another aspect of the invention, arrangement 900 may also include an emergency beacon 946 mounted in tower assembly 904, which is further mounted in console 903. The tower assembly and emergency beacon is similar to that shown in FIG. 7. Tower assembly 904 may be adapted to receive an electrical signal from the arithmetic processor 960. Emergency beacon 946 may be programmed to emit one or more visual, audio or palpable signals if a particular physiological indicator has reached a specified level and may be incorporated into any aspect of the invention disclosed herein. Arrangement 900 will further comprise an emergency button (not shown) adapted to energize the emergency beacon 946. The emergency button may also energize a signal to be relayed to a remote location, for example, a nursing station. The emergency button may be incorporated into any embodiment of the present invention disclosed herein.

Arrangement 900 may further include one or more audio visual mechanisms 945, including, but not limited to, at least one video camera adapted to transmit video images, speaker adapted to transmit audio speech, and a microphone adapted to receive audio speech. The audio visual mechanism 945 can also include a palpable mechanism as discussed above. Vital sign sensing device 905 may also be adapted to include a means for transmitting at least one of a video image or audio speech to a remote location. Audio visual mechanism 945 may be incorporated into any embodiment of the invention disclosed herein.

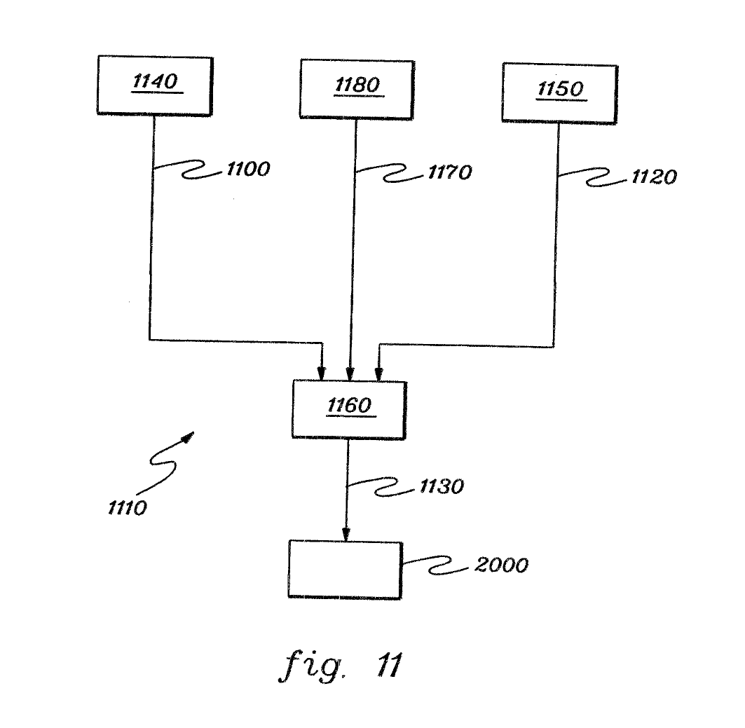

FIG. 10. shows a perspective view of vital sign sensing device 1095 according to another embodiment of the present invention. As shown in FIG. 10, device 1095 includes handgrip 1095 having finger indentations 1035 to guide a user's fingers (not shown) into placement on handgrip 1096 and for ease of gripping. Device 1095 is also adapted to include at least a first sensor 1040 and second sensor 1050 which are adapted to contact the hand of the user (not shown). First sensor 1040 and second sensor 1050 may include the same embodiment of first sensor 40 and second sensor 50 of FIG. 1.

According to FIG. 10, sensors 1040 and 1050 may be mounted on the surface of handgrip 1096. Handgrip 1095 may be cylindrical or non-cylindrical. Multiple physiological sensors may be mounted in handgrip 1095, including on finger indentations 1035.

First sensor 1040 and second sensor 1050 may be adapted to detect one or more vital signs of the user (not shown) and to output at least one electrical signal representative of the first vital sign, as discussed above. A third sensor 1080 may also be mounted in device 1095.

FIG. 11 shows a schematic diagram 1110 of an electrical system of the present invention. Diagram 1110 includes arithmetic processor 1160 of one of the vital sign sensing devices (not shown) disclosed herein. Arithmetic processor 1160 is adapted to receive electrical signals, for example, a first electrical signal 1110, second electrical signal 1120, and fourth electrical signal 1170, representative of vital signs. The first electrical signal 1110, second electrical signal 1120 and fourth electrical signal 1170 are emitted from vital sign sensors 1140, 1150, and 1180 respectively. For example, first sensor 1150 emits first electrical signal 1110. Second sensor 1150 emits electrical signal 1120 and third sensor 1180 emits electrical signal 1170. Arithmetic processor 1160 is further adapted to manipulate the electrical signals 1110, 1120 and 1170, and generate an additional electrical signal, such as third electrical signal 1130, representative of one or more vital signs. Electrical signal 1130 may typically be forwarded to device 2000 for various uses, for example, for storage, or for output such as to a screen printer or emergency beacon. The electrical signals 1110, 1120, 1170, and 1130 can be one of a wired or wireless signal. The wireless signal may include an RFID signal, a WIFI signal, a cellular signal, or other related signal. Arithmetic processor 1160 may be adapted to store one or more electrical signals corresponding to one or more vital signs and may further adapted to record the electrical signal(s) for one or more time periods. The electrical system disclosed in FIG. 11 may be included in any embodiment of the invention as disclosed herein.

It is noted that many variations of the embodiments described above may be utilized consistent with the present invention. Specifically, many features of the invention are optional.

While the above detailed description has shown, described, and pointed out novel features of the invention as applied to various embodiments, it will be understood that various omissions, substitutions, and changes in the form and details of the device or arrangements illustrated may be made by those skilled in the art without departing from the invention. The foregoing description is of the best mode presently contemplated of carrying out the invention. This description is in no way meant to be limiting, but rather should be taken as a mere illustration of the general principles of the invention. The scope of the invention should be determined with reference to the claims.

I claim:

1. A vital sign sensing device comprising:
a housing adapted to support a hand of a user, the housing comprising a cavity adapted to receive at least one finger of the user;
a first sensor mounted in the cavity, the first sensor adapted to detect a first vital sign of the user and to output a first electrical signal representative of the first vital sign;
a second sensor mounted on the housing, the second sensor adapted to contact the arm of the user and to detect a second vital sign of the user and to output a second electrical signal representative of the second vital sign; and
an arithmetic processor adapted to receive the first and second electrical signals, adapted to manipulate the first and second electrical signals, and adapted to generate a third electrical signal representative of at least one of the first and second vital signs.

2. The device as recited in claim 1, wherein the first sensor comprises a physiological indicator sensor.

3. The device as recited in claim 2, wherein the physiological indicator sensor comprises at least one of a pulse rate sensor, a pulse oxygenation sensor, a temperature sensor, an EKG sensor, a blood glucose sensor, a basal metabolism sensor, a body mass index sensor, and a body fat sensor or other physiological sensor.

4. The device as recited in claim 1, wherein the second sensor comprises a physiological indicator sensor.

5. The device as recited in claim 4, wherein the physiological indicator sensor comprises at least one of a pulse rate sensor, a pulse oxygenation sensor, a temperature sensor, an EKG sensor, a blood glucose sensor, a basal metabolism sensor, a body mass index sensor, and a body fat sensor or other physiological sensor.

6. The device as recited in claim 1, further comprising at least one third sensor, the third sensor adapted to detect a third vital sign of the user and to output a fourth electrical signal representative of the third vital sign.

7. The device as recited in claim 6, the at least one third sensor comprising a physiological indicator sensor.

8. The device as recited in claim 7, wherein the physiological indicator sensor comprises at least one of a pulse rate sensor, a pulse oxygenation sensor, a temperature sensor, an EKG sensor, a blood glucose sensor, a basal metabolism sensor, a body mass index sensor, and a body fat sensor or other physiological sensor.

9. The device as recited in claim 1, wherein the cavity comprises a cylindrical cavity having at least one aperture.

10. The device as recited in claim 1, wherein the cavity comprises a first cylindrical section and a second cylindrical section, the first section pivotally mounted to the second section.

11. The device as recited in claim 1, further comprising a display screen mounted in the housing.

12. The device as recited in claim 1 1, wherein the display screen is adapted to receive input from at least one of a user and a healthcare provider.

13. The device as recited in claim 1, wherein the device is mounted to a structure or piece of equipment.

14. The device as recited in claim 1, further comprising an arm cuff mounted to the housing, the arm cuff adapted to detect at least one of blood pressure and pulse rate.

15. The device as recited in claim 14, the arm cuff comprising an adjustable arm cuff.

16. The device a recited in claim 1, wherein the arithmetic processor is adapted to store at least one electrical signal corresponding to at least one vital sign.

17. The device as recited in claim 1, wherein the arithmetic processor is adapted to record the third electrical signal for at least one time period.

18-30. (canceled)

31. A console comprising the device as recited in claim 1.

32. (canceled)

33. An arrangement comprising the console as recited in claim 31 and a seating device.

34. (canceled)

35. A seating device comprising the device as recited in claim 1.

36-38. (canceled)

39. A console comprising a platform adapted to be inserted into a seating device and comprising a device as recited in claim 1.

40-58. (canceled)
